(19) United States
(12) Patent Application Publication
(10) Pub. No.: US 2013/0052098 A1
(43) Pub. Date: Feb. 28, 2013

(54) APPARATUS FOR STERILIZING AN INTERIOR PORTION OF AN INSTRUMENT

(71) Applicant: Neil KENNEDY, Carlsbad, CA (US)

(72) Inventor: Neil KENNEDY, Carlsbad, CA (US)

(73) Assignee: ALPHATEC SPINE, INC., Carlsbad, CA (US)

(21) Appl. No.: 13/589,153

(22) Filed: Aug. 19, 2012

Related U.S. Application Data
(60) Provisional application No. 61/529,033, filed on Aug. 30, 2011.

(51) Int. Cl. A61L 2/04

(52) U.S. Cl. 422/307

(57) ABSTRACT

An apparatus for sterilizing a medical instrument in a container by applying thermal energy to the container includes a first base member coupled to the container and an elongated member with a first end that couples to the first base member and a second end extending into an interior portion of the medical instrument. The first base member transfers a portion of the thermal energy to the elongated member to heat the interior portion.

### CROSS REFERENCE TO RELATED APPLICATIONS

The present application claims priority to U.S. Provisional Application No. 61/529,033, filed Aug. 30, 2011, entitled “Apparatus for Sterilizing an Interior of an Instrument” which is incorporated by reference in its entirety herein.

### FIELD

The present invention relates to relates to containers for sterilizing medical instruments, such as by heated steam from an autoclave, and more particularly to an apparatus for sterilizing an interior portion of an instrument.

### BACKGROUND

Sterilization of surgical instruments and the like is often performed in an autoclave where the instruments are exposed to steam at an elevated temperature. Typically, the instruments are placed in a container having a cover and a base. The surfaces of the cover and the base are perforated with holes to permit the steam to pass there through. The holes also ensure that the instruments within the container will be exposed to the hot steam immediately upon their introduction into the autoclave.

The steam includes an amount of thermal energy that transfers to the container and the instruments inside to increase a temperature on the surfaces of the instruments. Ideally, convective heat transfer from the steam to the instruments increases the surface temperature to a sterilization temperature to sterilize the surfaces of bacteria and contaminants. The temperature may be maintained at or above the sterilization temperature for a predetermined period of time to ensure complete sterilization of the surfaces of the instruments.

Some instruments include various interior portions having surfaces that are often difficult to sterilize. For example, medical devices such as handles for tools and other elongated, hollow instruments include channels, cannulae, and slotted portions that hamper the flow of the heated steam. Thus, while convective heat transfer to surfaces of exterior portions of the instruments may be sufficient, the convective heat transfer to these surfaces of interior portions may be limited because of the reduced flow of heated steam. Although some heat transfer may occur through conduction from the exterior portions to the interior portions, the problem becomes increasingly difficult when exterior portions of the instruments, such as handles, are over-molded with silicone or other thermal insulators for improving grip.

Current sterilization trays and containers rely almost entirely on sterilant flow (convection) to penetrate into these interior portions of the instruments. This results in costly delays for manufacturers and suppliers of medical instruments due to repeated sterilization testing.

### SUMMARY

An exemplary apparatus for sterilizing a medical instrument in a container by applying thermal energy includes a first base member coupled to the container and an elongated member with a first end that couples to the first base member and a second end extending into an interior portion of the medical instrument. The first base member transfers a portion of the thermal energy to the elongated member to heat the interior portion.

In other features, the first end is pivotably coupled to the first base member. The second end extends through the interior portion to a second base member coupled to the container. The first base member includes a thermally conductive material. The elongated member includes a thermally conductive material.

In still other features, the apparatus includes a locking member that increases contact between the elongated member and the first base member to increase thermal communication. The locking member includes a thermally resistive material.

In yet other features, the elongated member includes an interior portion having an opening for receiving heated steam. The elongated member conducts thermal energy to the interior portion of the medical instrument. The first base member receives thermal energy from heated steam by convective heat transfer. The first base member receives thermal energy from the container by conductive heat transfer. The first base member transfers thermal energy to the elongated member by conductive heat transfer. An interior portion of the elongated member receives thermal energy from heated steam by convective heat transfer. The first base member includes a removable base member for positioning in multiple locations within the container.

### BRIEF DESCRIPTION OF THE DRAWINGS

FIG. 1 is a perspective view of a sterilization container.

FIG. 2 is a top view of a base portion of the sterilization container including an exemplary sterilization apparatus according to the principles of the present disclosure.

FIGS. 3 and 4 illustrate perspective views of the exemplary sterilization apparatus and a cannulated handle according to the principles of the present disclosure.

FIGS. 5A and 5B are side views of the exemplary sterilization apparatus according to the principles of the present disclosure.

FIGS. 6A and 6B are side views of the exemplary apparatus including a cannulated handle in a loading position and a sterilizing position according to the principles of the present disclosure.

FIG. 7 is a top view of the exemplary sterilization apparatus according to the principles of the present disclosure.

FIG. 8 is a cross-sectional view of the exemplary sterilization apparatus according to the principles of the present disclosure.

FIG. 9 is a schematic view illustrating convective heat transfer to exterior and interior portions of an instrument.

FIG. 10 is a schematic view illustrating convective and conductive heat transfer to exterior and interior portions of the instrument via the exemplary sterilization apparatus according to the principles of the present disclosure.

### DETAILED DESCRIPTION

Embodiments of the invention will now be described with reference to the Figures, wherein like numerals reflect like elements throughout. The terminology used in the description presented herein is not intended to be interpreted in any limited or restrictive way, simply because it is being utilized in conjunction with detailed description of certain specific embodiments of the invention. Furthermore, embodiments of the invention may include several novel features, no single one of which is solely responsible for its desirable attributes or which is essential to practicing the invention described herein. The words proximal and distal are applied herein to denote specific ends of components of the instrument described herein. A proximal end refers to the end of an instrument nearer to an operator of the instrument when the instrument is being used. A distal end refers to the end of a component further from the operator and extending towards the surgical area of a patient and/or the implant.

The apparatus of the present invention includes features that enable improved heat transfer to the interior portion of medical instruments for sterilization within a container. The apparatus includes a base member that couples to the container and an elongated member with a first end that couples to the first base member. A second end of the elongated member extends into an interior portion of the medical instrument. The first base member transfers a portion of the thermal energy from surrounding steam to the elongated member to heat the interior portion.

The apparatus of the present invention is advantageous over the prior art for various reasons. The apparatus reduces the amount of time required to heat the interior portion of the instrument to the sterilization temperature. The apparatus improves the transfer of thermal energy to the interior portion by utilizing conductive heat transfer and relying less on the convective heat transfer associated with sterilant flow. Conductive heat transfer may be applied to a variety of interior portions of instruments by selecting various shapes and lengths of elongated members.

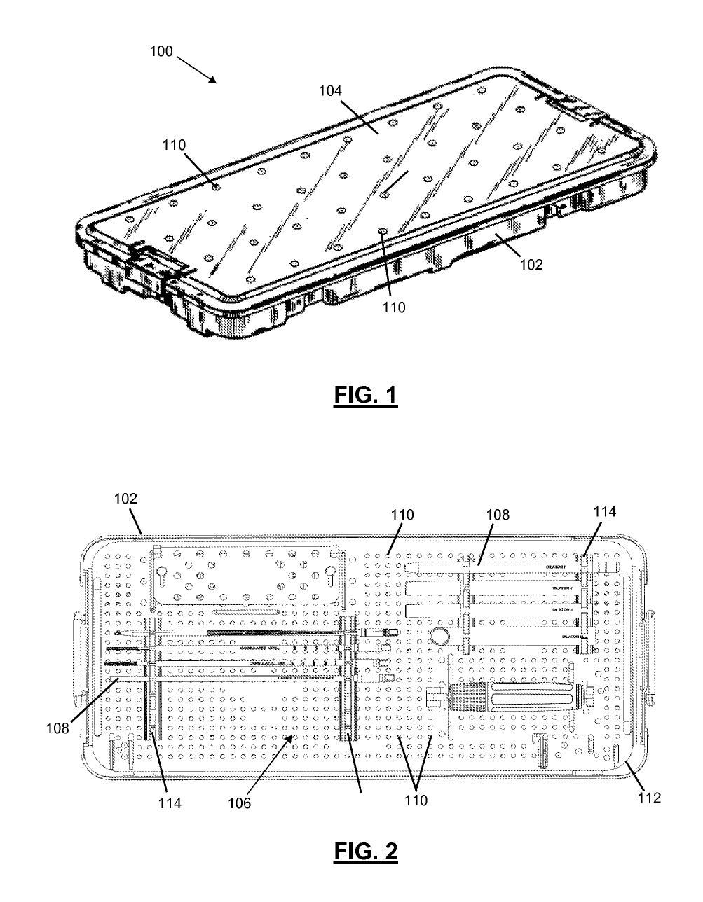

Referring now to FIGS. 1 and 2, a sterilization container 100 includes a base 102 and a removable cover 104. The base 102 and cover 104 define an interior cavity 106 for the sterilization of various medical and surgical instruments 108. Typically, an autoclave may be used to sterilize equipment and supplies, such as the instruments 108, by subjecting the container 100 to high pressure saturated steam at approximately 121° C. for around 15-20 minutes depending on the size of the load and the contents. Sterilization of the instruments 108 generally refers to increasing the temperature of the surfaces of the instruments 108 to a sterilization temperature. The sterilization temperature may be approximately equal to or slightly less than the temperature of the saturated steam. Generally, thermal energy from the steam transfers to the surfaces of the instruments 108 via convective heat transfer. The heat transfer process may require a period of time to increase the surface temperatures to the sterilization temperature.

The case 102 and cover 104 include holes 110 to enable the steam to pass through the container 100 to sterilize instruments 108 within the interior cavity 106. The instruments 108 may be mounted onto various trays 112 within the container 100. For example, some trays 112 may include fasteners 114 that clamp around the exterior of the instruments 108. Some trays 112 may include shaped sections formed along an outline of exterior portions of the instruments 108. The trays 112 likewise include holes 110 to allow steam to pass there through. Multiple trays 112 may stack one upon another inside the container 100. As the steam passes through the holes 110 into the cavity 106, thermal energy from the steam may transfer to the base 102, the cover 104, the instruments 108, and the trays 112 by convective heat transfer. The heat transfer increases temperatures on various surfaces of the instruments 108.

As the steam travels towards the center of the container 100, the amount of thermal energy dissipates due to some of the heat transferring to the container 100, instruments 108, and the tray 112. The thermal energy of the steam may continue to dissipate until the temperatures of the various heated components reaches the sterilization temperature of the steam and thus, thermal equilibrium with the steam. Therefore, the autoclave must continue to supply the steam for a predetermined period of time depending on thermal characteristics of the container 100, trays 112, and instruments 108. For example, portions of instruments 108 nearer to holes 110 on a perimeter of the container 100 may reach thermal equilibrium in less time than instruments 108 nearer the center of the container 100.

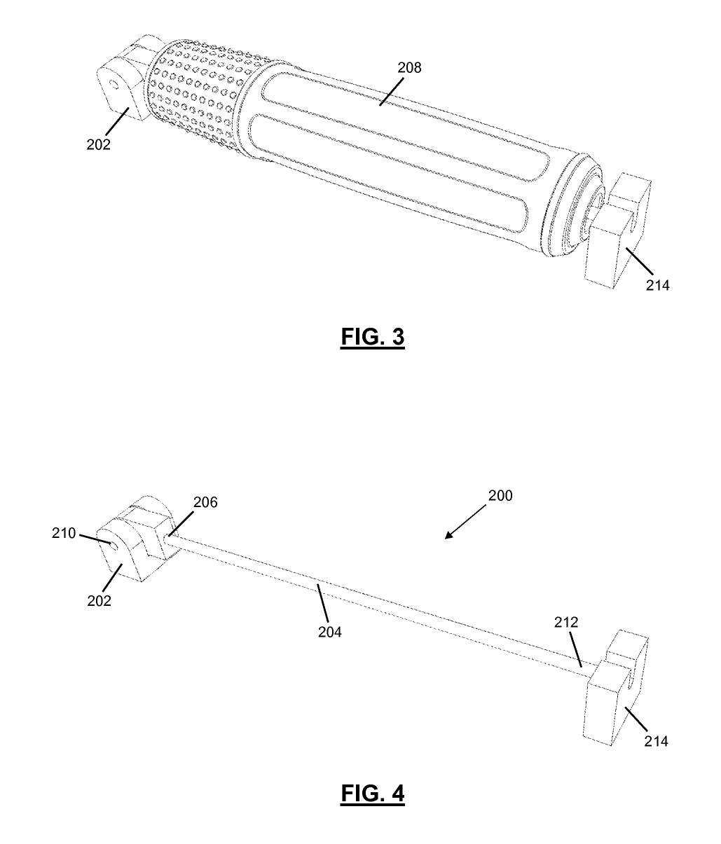

Referring now to FIGS. 3 and 4, an exemplary sterilization apparatus 200 for sterilizing an instrument such a handle 208 is shown. The apparatus 200 may be used in conjunction with the container 100 and an autoclave or other device that applies thermal energy to the container 100 such as with heated steam. The apparatus 200 includes a first base member 202 that attaches to the tray 112 and a thermally conductive member 204 that couples with the first base member 202 at a first end 206. The first base member 202 may be removable from the container 100 to enable multiple configurations for a variety of instruments. The first base member 202 and the conductive member 204 may be comprised of any material with relatively low thermal resistivity (i.e. relatively high thermal conductivity) to enable faster heating and transfer of heat during the sterilization process. For example, the material may comprise aluminum, copper, zirconium, and other metals or alloys of such metals.

The conductive member 204 may be an elongated member configured to conduct heat from the first base member 202 inside of the handle 208. For example, the conductive member 204 may be configured as an elongated pin that extends into the center of the handle 208 as illustrated in FIG. 8. In other examples, the conductive member 204 may include a flexible wire or coil capable of bending to various shapes corresponding to interior portions or cavities of the handle 208 or other instrument 108. Ideally, the thinner the conductive member 204 is, the more quickly it will increase to the sterilization temperature. However, the thinner the conductive member 204 is, the more fragile and likely to fail over an extended period of use. A coil may also expand and contract more easily to better fill some interior portions and cavities and release upon cooling.

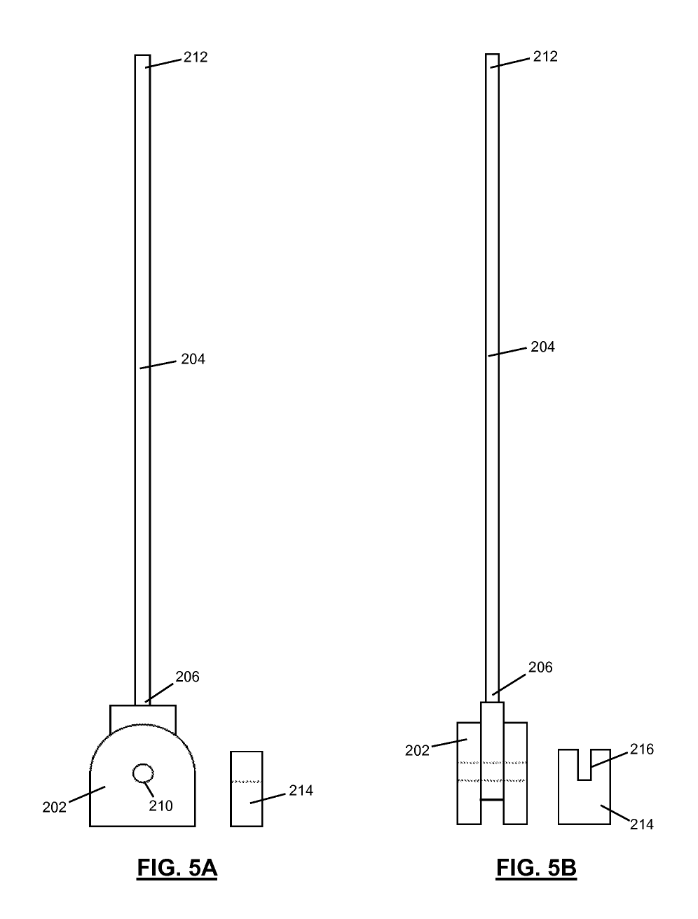

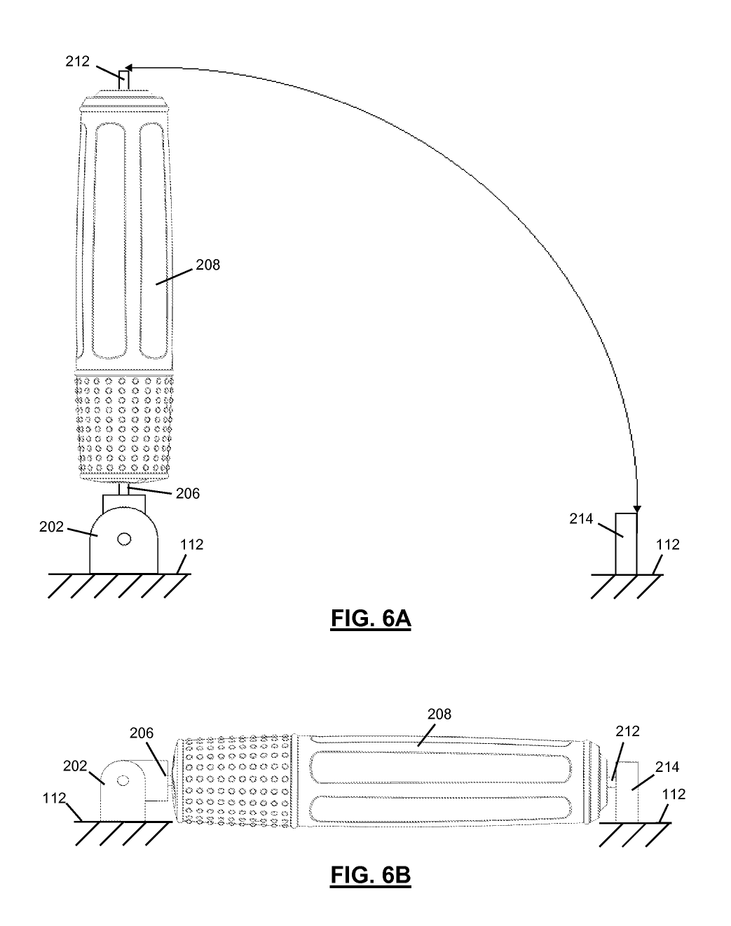

The conductive member 204 may be pivotably coupled to the first base member 202 by a pin 210. For example, the conductive member 204 may pivot to a vertical loading position shown in FIGS. 5A and 5B. In FIG. 6A, the handle 208 may slide over a second end 212 of the conductive member 204. The second end 212 of the conductive member 204 may extend through the handle 208 and protrude outside the handle 208. As the conductive member 204 pivots into a horizontal sterilization position shown in FIG. 6B, the second end 212 may couple with a second base member 214 that attaches to the tray 112. The second base member 214 may also be comprised of any material with relatively low thermal resistivity similar to the first base member 202.

To ensure maximum conductive heat transfer, the first and second ends 206 and 212 of the conductive member 204 should directly contact as much of the first and second base members 202 and 214 as possible. A locking member, such as a clamp (not shown), may compress the first end 206 into greater contact with the first base member 202 and pin 210 to maximize conductive heat transfer between engaging surfaces. The second end 212 may lock within a receiver portion 216 of the second base member 214. Another locking member (also not shown) may compress the second end 999 into greater contact with the second base member 214 to maximize conductive heat transfer between engaging surfaces.

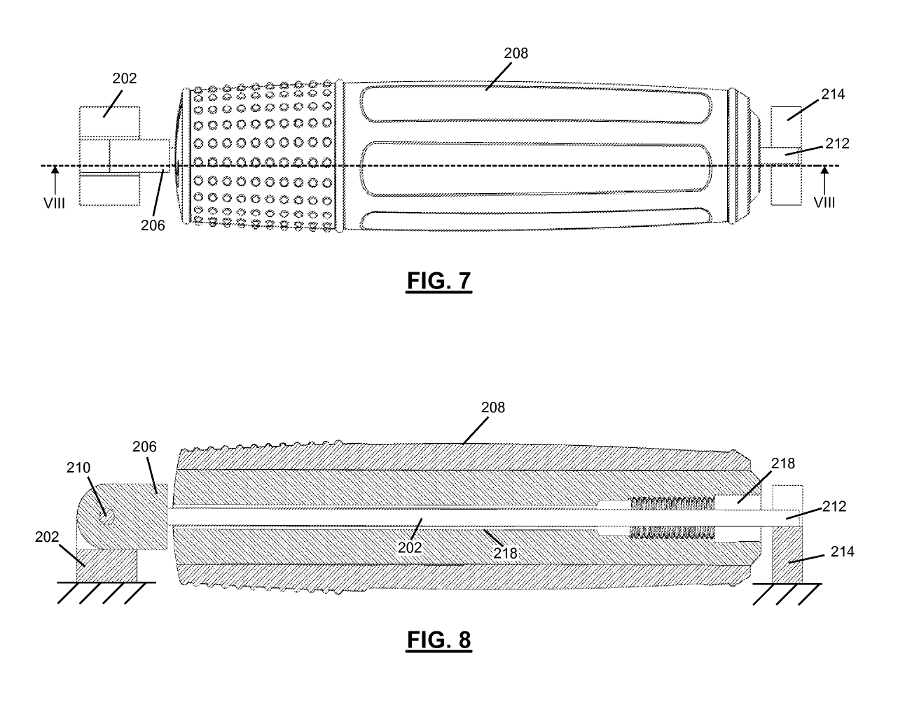

FIG. 8 illustrates a cross-sectional view of the handle 208 and sterilization apparatus 200 in FIG. 7. In FIG. 8, the conductive member 204 passes through a cannula 218 in the handle 208. The cannula 218 may contact a portion of the conductive member 204. For example, the handle 208 may tend to hang from the conductive member 204 in the sterilization position. The surface of the cannula 218 may contact the surface of the conductive member 204. The conductive member 204 may be configured to slidably engage the cannula 218 prior to increasing to the sterilization temperature. As the temperature increases, the conductive member 204 may expand to contact more of the surface of the cannula 218. Increasing contact between the conductive member 204 and the surface of the cannula 218 improves heat transfer to the surface to reduce sterilization times.

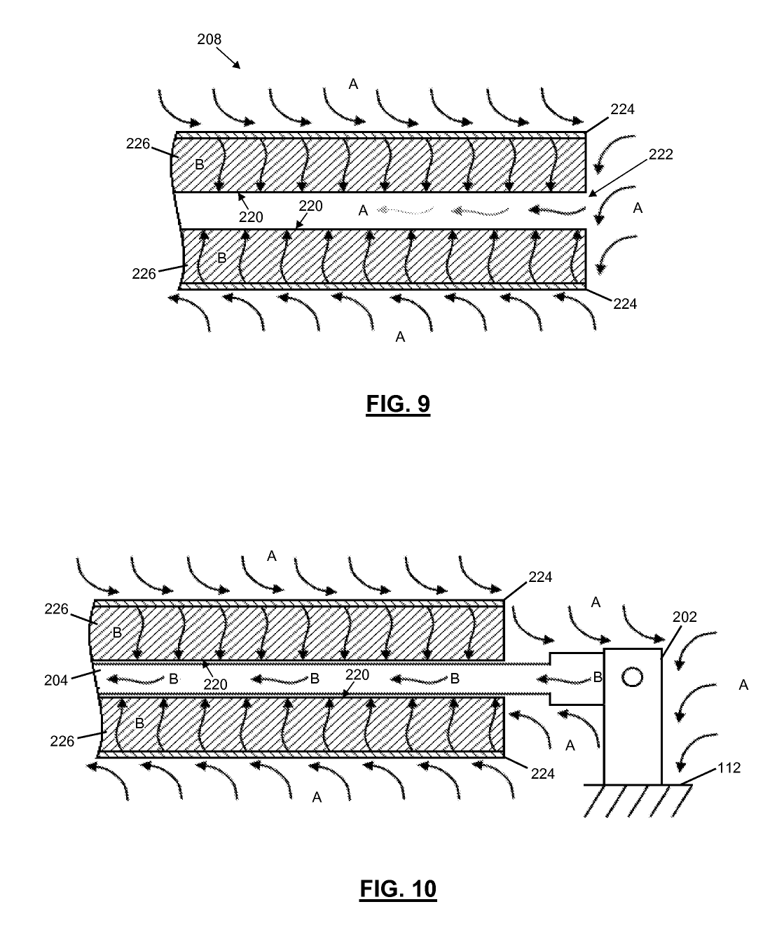

Referring now to FIGS. 9 and 10, heat transfer may be illustrated schematically using arrows A for convective heat transfer and arrows B for conductive heat transfer. In FIG. 9, a cannulated instrument, such as the handle 208, may include an interior surface 220 that may be difficult to sterilize using solely convective heat transfer from the high pressure steam. For example, some of the steam may enter the interior of the handle 208 through an opening 222 at the end of the cannula 210. The steam may transfer some heat to the interior surface 220 through convection. However, the size of the opening 222 restricts flow of steam into the interior of the handle 208.

Some of the steam may also surround the exterior surface 224 of the handle 208 and transfer some heat to the exterior surface 224 by convection A. The temperature of the exterior surface 224 heats up and additional heat may transfer to the interior surface 220 through conduction B via a substrate 226 of the handle 208. For example, the substrate 226 may comprise various thermally conductive materials. However, many instruments, such as handles 208, may also include a silicone or rubber grip on the exterior surface 224 comprised of a thermally resistive material that decreases. Thus, the exterior surface 224 may decrease the amount of heat transferred to the interior surface 220 through conduction.

In FIG. 10, the sterilization apparatus 200 increases heat transfer to the inner surface 220 using conduction through the conductive member 204. For example, the steam may transfer heat to the first base member 202 and conductive member 204 by convection thus increasing a temperature of the conductive member 204. The conductive member 204, which extends into the interior of the handle 208, then transfers heat to the interior surface 220 by convection, radiation, and/or conduction. Thus, the elongated member 204 provides a conduit for multiple forms of heat transfer to heat the interior surface 220 of the instrument 208 to the sterilization temperature more quickly than by a single form of heat transfer as shown in FIG. 9.

Example embodiments of the methods and apparatus of the present invention have been described herein. As noted elsewhere, these example embodiments have been described for illustrative purposes only, and are not limiting. Other embodiments are possible and are covered by the invention. Such embodiments will be apparent to persons skilled in the relevant art(s) based on the teachings contained herein. Thus, the breadth and scope of the present invention should not be limited by any of the above-described exemplary embodiments, but should be defined only in accordance with the following claims and their equivalents.

The invention claimed is:

1. An apparatus for sterilizing a medical instrument in a container by applying thermal energy, comprising:
a first base member coupled to the container; and
an elongated member with a first end that couples to the first base member and a second end extending into an interior portion of the medical instrument;
wherein the first base member transfers a portion of the thermal energy to the elongated member to heat the interior portion.

2. The apparatus of claim 1, wherein the first end is pivotably coupled to the first base member.

3. The apparatus of claim 1, wherein the second end extends through the interior portion to a second base member coupled to the container.

4. The apparatus of claim 1, wherein the first base member includes a thermally conductive material.

5. The apparatus of claim 1, wherein the elongated member includes a thermally conductive material.

6. The apparatus of claim 1, further comprising a locking member that increases contact between the elongated member and the first base member to increase thermal communication.

7. The apparatus of claim 6, wherein the locking member includes a thermally resistive material.

8. The apparatus of claim 1, wherein the elongated member includes an interior portion having an opening for receiving heated steam.

9. The apparatus of claim 1, wherein the elongated member conducts thermal energy to the interior portion of the medical instrument.

10. The apparatus of claim 1, wherein the first base member receives thermal energy from heated steam by convective heat transfer.

11. The apparatus of claim 1, wherein the first base member receives thermal energy from the container by conductive heat transfer.

12. The apparatus of claim 1, wherein the first base member transfers thermal energy to the elongated member by conductive heat transfer.

13. The apparatus of claim 1, wherein an interior portion of the elongated member receives thermal energy from heated steam by convective heat transfer.

14. The apparatus of claim 1, wherein the first base member includes a removable base member for positioning in multiple locations within the container.
